(19) United States
(12) Patent Application Publication
(10) Pub. No.: US 2010/0048692 A1
(43) Pub. Date: Feb. 25, 2010

(54) Naphthofuranone Derivatives as Specific Inhibitors of Thymidylate Synthases

(76) Inventors: Alberto Venturelli, Gorzano di Maranello (IT); Maria Paola Costi, Modena (IT); Piergiorgio Pecorari, Rubiera (IT); Tiziana Rossi, Modena (IT); Chiara Casolari, Modena (IT); Donatella Tondi, Modena (IT); Daniela Barlocco, Milano (IT)

Correspondence Address: DILWORTH & BARRESE, LLP, 1000 WOODBURY ROAD, SUITE 405, WOODBURY, NY, 11797, US

(21) Appl. No.: 12/309,018

(22) PCT Filed: Jul. 3, 2007
(86) PCT No.: PCT/EP2007/006006

§ 371 Date: Oct. 13, 2009

(30) Foreign Application Priority Data
Jul. 5, 2006 (IT) VA/2006/A 000039

(51) Int. Cl. A61K 31/343; C07D 307/00

(52) U.S. Cl. 514/468; 549/299

(57) ABSTRACT

Synthetic compounds of 1,2-naphthalein moelcules (I) having specific inhibitory properties of the enzymatic activity of thymidylate synthases of bacterial species, their preparation, their pharmaceutical composition and use in the treatment and prophylaxis of infectious pathologies are disclosed.

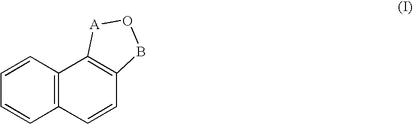

### FIELD OF THE INVENTION

The present invention is to be in the field of pharmaceutical chemistry and it aims at 1,2 naphthalein derivative compounds; their use as specific inhibitors of bacterial thymidylate synthase (TS) and of enzymes which are significantly similar and present in organisms of different origins.

The compounds which are hereafter described present high therapeutical usefulness as they constitute drugs for treatment, prevention and prophylaxis of infectious pathologies in superior animals particularly for what concerns human beings.

This invention deals with 1-2 naphthalein compounds having the following general formula (I):

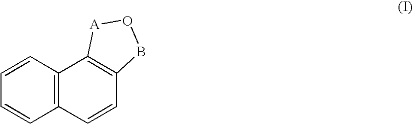

Wherein: A and B represent C═O, or a molecular fragment with formula G:

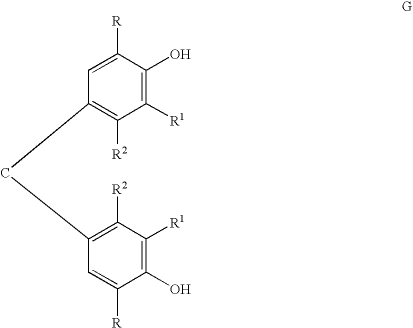

Wherein: R R¹ R² can be independently as:
(a) hydrogen
(b) C₁₋₁₂ linear or branched alkyl chain, which can alternatively contain one to three double or triple bonds and furthermore can optionally be replaced by one to two or three substituents like fluorine, chlorine, bromine, iodine, NO₂, NR₂³ where R³ can be hydrogen, C₁₋₁₂ alkyl.
(d) halogen, such as fluorine, chlorine, bromine, iodine,
(e) NO2, NR2, SH, SO3HR3, where R³ is as previously defined;

With the proviso that when A is C═O, B is G, and when A is G, then B is C═O.

Moreover are hereafter described the method of preparation of these compounds, the relative pharmaceutical composition and their use as bacterial TS inhibitors for treatment, prevention and prophylaxis of infectious pathologies.

As far as experts can see, the general formula (I) previously delineated relates independently to regioisomeric compounds having structure Iα and, respectively, (Iβ):

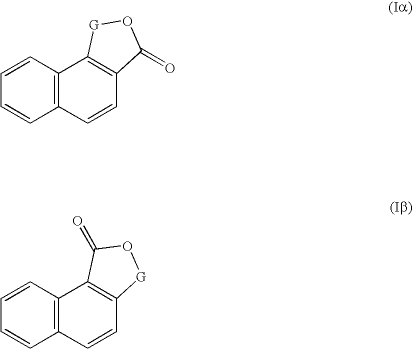

where G appears to be the same as described before. Secondly, to each of regioisomeric compounds having structure (Iα) e (Iβ) correspond, as a consequence of specific meanings of R, R¹ and R², enantiomers, diastereoisomers or different geometrical isomers both pure compounds and in mixtures.

This general formula (I) intends to describe all of the possible enantiomers, diastereoisomers or geometrical isomers, both individually and in mixture.

### BACKGROUND OF THE INVENTION

Thymidylate synthase (TS) is an enzyme whose basic function is to produce the catalysis of the methylation reaction relating to the 2′-deoxyuridine-5′-monophosphate (dUMP) to the 2′-deoxyuridine-5′monophosphate (dTMP)), that then is phosphorilated by proper kinases, and with this form it enters as essential element into DNA.

TS enzyme, in the isoenzyme form, is found in cells belonging to almost every living organism, included mammals, bacteria and pathogenous or non pathogenous parasites.

As TS inhibition causes cellular death, the control or the block of specific TS enzymatic activity given by proper inhibitor compounds can produce a considerable therapeutical potential in the treatment of hyperproliferative pathologies. E.g., human TS (hTS) is now recognized as therapeutic target for anticancer therapy, but, because of the structural identity between cancerous cells TS and healthy cells TS, the therapeutical index of used drugs seems to be low with, consequently, unwanted side effects.

Similar folate derivative compounds, as human TS inhibitors for anticancer use, are described, e.g., in:
Kisliuk R L., Deaza analogs of folic acid as antitumor agents. Curr Pharm Des. 2003;9(31):2615-25.
Purcell W T, Ettinger D S. Novel antifolate drugs. Curr Oncol Rep. 2003 March;5(2):114-25.
Costi M P, Ferrari S. Update on antifolate drugs targets. Curr Drug Targets. 2001 June;2(2):135-66.
Takimoto C H. Antifolates in clinical development. Semin Oncol. 1997 October;24(5 Suppl 18):S18-40-S18-51.
Boger D L, Labroli M A, Marsilje T H, Jin Q, Hedrick M P, Baker S J, Shim J H, Benkovic S J. Conformationally restricted analogues designed for selective inhibition of GAR Tfase versus thymidylate synthase or dihydrofolate reductase. Bioorg Med Chem. 2000 May;8(5): 1075-86.
Hart B P, Haile W H, Licato N J, Bolanowska W E, McGuire J J, Coward J K. Synthesis and biological activity of folic acid and methotrexate analogues containing L-threo-(2S,4S)-4-fluoroglutamic acid and DL-3,3-difluoroglutamic acid. J Med Chem. 1996 Jan. 5;39(1):56-65.
McGuire J J. Anticancer antifolates: current status and future directions. Curr Pharm Des. 2003;9(31):2593-613.
Kamen B. Folate and antifolate pharmacology. Semin Oncol. 1997 October;24(5 Suppl 18):S18-30-S18-39.
Thorndike J, Kisliuk R L, Gaumont Y, Piper J R, Nair M G. Tetrahydrohomofolate polyglutamates as inhibitors of thymidylate synthase and glycinamide ribonucleotide formyltransferase in Lactobacillus casei. Arch Biochem Biophys. 1990 March;277(2):334-41.
Bertino J R, Sobrero A, Mini E, Moroson B A, Cashmore A. Design and rationale for novel antifolates. NCI Monogr. 1987;(5):87-91.
Pignatello R, Spampinato G, Sorrenti V, Di Giacomo C, Vicari L, McGuire J J, Russell C A, Puglisi G, Toth I. Lipophilic methotrexate conjugates with antitumor activity. Eur J Pharm Sci. 2000 May;10(3):237-45.
Faessel H M, Slocum H K, Jackson R C, Boritzki T J, Rustum Y M, Nair M G, Greco W R. Super in vitro synergy between inhibitors of dihydrofolate reductase and inhibitors of other folate-requiring enzymes: the critical role of polyglutamylation. Cancer Res. 1998 Jul. 15;58(14):3036-50.
Chen V J, Bewley J R, Andis S L, Schultz R M, Iversen P W, Shih C, Mendelsohn L G, Seitz D E, Tonkinson J L. Cellular pharmacology of MTA: a correlation of MTA-induced cellular toxicity and in vitro enzyme inhibition with its effect on intracellular folate and 7 nucleoside triphosphate pools in CCRF-CEM cells. Semin Oncol. 1999 April;26(2 Suppl 6):48-54.
Calvert H. Folate status and the safety profile of antifolates. Semin Oncol. 2002 April;29(2 Suppl 5):3-7.
Shih C, Chen V J, Gossett L S, Gates S B, MacKellar W C, Habeck L L, Shackelford K A, Mendelsohn L G, Soose D J, Patel V F, Andis S L, Bewley J R, Rayl E A, Moroson B A, Beardsley G P, Kohler W, Ratnam M, Schultz R M. LY231514, a pyrrolo[2,3-d]pyrimidine-based antifolate that inhibits multiple folate-requiring enzymes. Cancer Res. 1997 Mar. 15;57(6):1116-23.
Chen V J, Bewley J R, Andis S L, Schultz R M, Iversen P W, Shih C, Mendelsohn L G, Seitz D E, Tonkinson J L. Preclinical cellular pharmacology of LY231514 (MTA): a comparison with methotrexate, LY309887 and raltitrexed for their effects on intracellular folate and nucleoside triphosphate pools in CCRF-CEM cells. Br J Cancer. 1998;78 Suppl 3:27-34.
Newell D R. Clinical pharmacokinetics of antitumor antifolates. Semin Oncol. 1999 April;26(2 Suppl 6):74-81.
Jones T R, Smithers M J, Taylor M A, Jackman A L, Calvert A H, Harland S J, Harrap K R. Quinazoline antifolates inhibiting thymidylate synthase: benzoyl ring modifications. J Med Chem. 1986 April;29(4):468-72.

None of the previous sources refers to any compounds, use of compounds, or methods capable of inhibiting bacterial TS. Nowadays there are no specific known inhibitors for bacterial or parasitic TS which can be used as drugs.

Indeed, also isoenzymatic forms of TS relating to different groups of bacteria and parasites might be potentially considered an useful therapeutical target to reach in order to treat infectious pathologies affecting mammals; especially in regard to the fact that therapeutical needs are constantly increasing as a consequence of the appearance of several bacterial pathogenic families which resulted highly resistant, because they are lowering the effectiveness of the drugs currently used. As the number of bacteria and parasites resistant to currently used drugs which are employed during therapy is constantly increasing, the needs for the identification of new biological target and their specific inhibition are becoming more and more urgent.

Because of the fact that TS is one of the most conserved enzymes in the species evolution, several efforts are being made in order to identify compounds which could specifically or properly inhibit TS of pathogenic species, producing little/no effects on mammal TS; this researching activity may possibly provide new tools against infectious diseases, even if no result presenting any practical application has been obtained yet.

The present inventors have recently succeeded in giving a complete description of 2,3 and 1,8 naphthalein derivative compounds as TS inhibitors; some of these substances resulted to be capable of inhibiting through species-specificity the bacterial TS related to non-pathogenic Lactobacillus casei (Costi M P, Rinaldi M, Tondi D, Pecorari P G, Barlocco D, Ghelli S, Stroud R M, Santi D V, Stout T J, Musiu C, Marangiu E M, Pani A, Congiu D, Loi G A, La Colla P. Phthalein derivatives as a new tool for selectivity in thymidylate synthase inhibition. J. Med. Chem. 42, 2112-2124; 1999).

Consequently, the realization of compounds capable of specific inhibition of the bacterial TS with respect to the mammalian one, particularly the human one, appears to be an highly desirable target because it could actually represent a notable therapeutical progress in the treatment of bacterial infections, especially the resistant ones; it is worth considering the importance of obtaining new drugs which present a high therapeutical index and can result to be useful to the treatment of bacterial infections resisting to currently therapeutical agents.

### DESCRIPTION OF THE INVENTION

The present inventors have now identified, throughout detailed and accurate researches aiming at the identification of specific TS inhibitor compounds, new 1,2-naphthalein substances having the general formula (I), which actually represent the main aspect of the present invention. The above mentioned compounds specifically inhibit bacterial TS and, surprisingly, they can deal with pathogenic bacteria TS, not properly with the mammalian TS, in particularly the human one.

Several compounds belonging to the same 1,2-naphthalein family have already been prepared and described, e.g.:
Roschger P. 1,2-Naphthaloperinone dyes for the bulk dyeing of plastics, their preparation and use, EP0570800.
Masahiro M. Mitsuhisa Y. Process for preparing substituted aromatic compounds and intermediates thereof, WO02/34712.
Karl D.; Lathia, Dinesh; Nolte, Wilfried; Roeker, Klaus D. Molecular structure and chemiluminescence. VI. Influence of the positions of substituents on the chemiluminescence of benzo[f]phthalazine-1,4(2H,3H)-diones. Justus Liebigs Ann. Der Chemie (1974), (5), 798-808.
Dozen, Yasuhiko. Syntheses of all isomers of naphthalenetricarboxylic acids. Bull Chem Soc Japan (1972), 45(2), 519-25.
Newman, Melvin S.; Gaertner, Russell. The synthesis of polynuclear aromatic hydrocarbons. I. Methyl-1,2-benzanthracenes. J Amer Chem Soc (1950), 72 264-73.

Some of the disclosed products have just been tested especially for their laxative properties (Laxatives: chemical structure and potency of phthaleins and hydroxyanthraquinones. Hubacher, Max H.; Doernberg, Sidney; Horner, Arthur. Ex-Lax., Inc., Brooklyn, N.Y., J Amer Pharmac Assoc (1912-1977) (1953), 42 23-30; Laxative activity of triphenylmethane derivatives. I. Relationship between structure and activity of phenolphthalein congeners. Loewe, S. J Pharmacol Exp Ther (1948), 94 288-98).

None of the previous references is supposed to deal with an exhaustive description of the present compounds, neither any antibiotic activity proper of 1,2 naphthalein compounds is mentioned.

The compounds included in the present invention show an antibiotic activity towards pathogenous bacteria that could be seen both IN VITRO and IN VIVO, and so are potentially useful to the treatment and/or the prophylaxis of bacterial infections that hit mammals, most of human beings.

Another basic aspect related to this invention is that TS inhibitor compounds of general formula (I) can be separated by TS inhibitors known in the “ART” in that they are species—specific, and so they can be used in order to inhibit bacterial TS but not mammalian TS particularly human TS; in this way they result potentially without any unwished collateral effect when they are given to a mammal patient, most of all a human, who requires antibacterial treatment, prevention and/ or prophylaxis.

Another side of the present invention is the method followed to prepare compounds relating to formula (I), that includes the steps of:
causing the 1,2-naphthalic anhydride to react with a phenol compound belonging to general formula

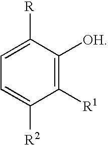

where R, R¹ and R² are, as previously described, in molar ratio at least of 1:2, or higher in order to give a mixture composed by two regioisomers of formula (Iα) and, respectively, (Iβ), where “G ” is reported as described before.

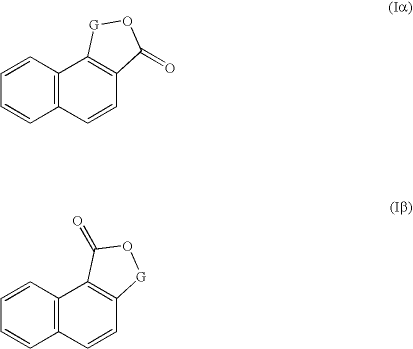

In this mixture the percentage of each regioisomer can be included between 0% and 100%, since this quantity is depending on the peculiar meanings that has the nature of R, R¹ and R² substituents, on the phenolic ring, and of the environment where the reaction takes place; this ratio is also related to the separation and the purification of every regioisomer that has formula (Iα) and, respectively, (Iβ) from the mixture.

Furthermore it is possible to turn later on a regioisomeric compound that has formula (Iα), obtained as previously described, into another compound that has formula (Iα) and a regioisomeric compound having formula (Iβ) into another compound of formula (Iβ). This reaction between 1,2-naphthalic anhydride and a phenol compound can be carried out in acidic conditions, e.g. in the presence of an amount of an acid that can vary from catalytic to three molar equivalents, as regards to naphthalic anhydride, without or with an inert solvent, at a temperature between about 50° C. and about 250° C. Suitable acids that can be used are: protic acids as sulphuric concentrated acid, or Lewis' acids, as stannic tetrachloride (SnCl₄) and aluminium trichloride (AlCl3). Favourite reaction conditions are, e.g., in the presence of 5-6 drops of H2SO₄ at a temperature included between nearly 180° C. and 190° C., or in the presence of 0.40-1.20, more properly 0.6-0.8, molar equivalent of SnCl₄, or in the presence of 1.2-2.6 molar equivalent of AlCl3, at a temperature included between about 110° C. and 120° C.

The further conversion of a regioisomeric compound that has formula (Iα) into another compound that has formula (Iα), or the conversion of a regioisomer compound that has formula (Iβ) into another compound that has formula (Iβ) can be realized using conventional methods known from synthetic organic chemistry, e.g. as those described in H. 0. House, Modern Synthetic Reactions, Benjamin, Menlo Park (USA); J. March, Advanced Organic Chemistry, John Wiley & Sons, Chichester (GB); R. C. Larock, Comprehensive Organic Transformations, VCH Verlagsgesellschaft mbH, Weinheim (D).

Examples of favourite compounds belonging to the present invention are compounds of formula (Iα)

| | |
|---|---|
| | (Iα) |
| 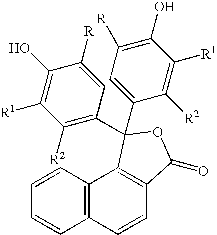 | |

| Compound | | | |
|---|---|---|---|
| N^{o} | R | R¹ | R² |
| 1 | H | H | H |
| 2 | CH(CH₂)₂ | H | CH₃ |
| 3 | I | H | H |
| 4 | Br | H | H |
| 5 | Br | Br | H |

and compounds of formula (Iβ)

| | |
|---|---|
| | (Iβ) |
| 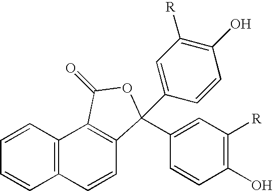 | |

| Compound | |
|---|---|
| N^{o} | R |
| 6 | H |
| 7 | Cl |
| 8 | F |

A remarkable aspect of the present invention includes the usage of an effective amount relating to a compound that has general formula (I), as previously defined, in order to prepare a medicament for the treatment, the prevention or the inhibition of an infectious pathology caused by bacteria; the treatment of this contagious pathology can be executed or simplified by inhibiting the thymidylate synthase activity of pathogenous bacteria, including (but non restricted to), Enterococcus faecalis, Staphilococcus aureus, Criptococcus neoformans, Pneumocistis carinii, Listeria monocytogenes, Streptococcus spp., in a mammals, particularly human beings.

A further fundamental aspect of the present invention embraces also a pharmaceutical composition including a quantity of a compound that has general formula (I), as previously defined, that will become effective for the treatment, the prevention or the inhibition of an infectious pathology, as described before, and an acceptable pharmaceutical carrier.

Therefore, the compound can be formulated for oral or parenteral administration to the therapeutical or prophylactic treatment of bacterial infections.

As a specific feature, the present compound, according to this invention (active substance), is to be mixed with conventional pharmaceutical carriers and excipients and can be used in the formulation of tablets, capsule, suspensions, syrups, and others. Such pharmaceutical compositions are likely to contain 0.1 to 90% by weight of their active compound, and generally from 10 to 30% by weight. Pharmaceutical compositions can contain ordinary carriers and excipients, such as maize starch, lactose, sucrose, microcrystalline cellulose, caolin, mannitol, dicalcium phosphate, sodium chloride and alginic acid. Disintegrators usually used in the formulations of the present invention include microcrystalline cellulose, maize starch, and sodium starch glycolate and alginic acid.

A liquid composition may generally consist of a suspension or solution of the compound in a suitable liquid carrier or carriers, for example ethanol, glycerine, sorbitol, non aqueous solvent as polyethylene glycol, oleum or water with suspending agent, preservative, surfactant, wetting agent, flavouring or colouring agents.

Otherwise, any liquid formulation can be obtained using a reconstitutive powder. E.g. a powder containing the active compound, the suspending agent, sucrose and a sweetener might be reconstituted with water. furthermore a syrup may be prepared using a specific powder which contains the active compound, sucrose and sweetener.

A composition in the form of tablet might be prepared by using any suitable carrier or carriers usually used for solid formulations. Examples of such carriers include magnesium stearate, starch, lactose, sucrose, microcrystalline cellulose and ligands, e.g. povidone.

Where appropriate, the tablets may be prepared with coatings, coloured if desired, with enteric coatings or as to provide controlled release of active ingredients in the intestinal tract.

Moreover, the active ingredient can be formulated as to provide controlled release of active ingredients as tablet included in a hydrophilic or hydrophobic matrix.

A composition in the form of capsule can be prepared using ordinary procedures of incapsulation, e.g including the active ingredient and excipients in one capsule of hard gelatine.

Alternatively a semi-solid matrix of an active ingredient and polyethylene glycol of high molecular weight can be prepared and inserted in a capsule of hard gelly; or a solution of the active ingredient in polyethylene glycol or in an suspension of edible oil, e.g. liquid paraffin or fractioned coconut oil, can be prepared and inserted in a capsule of soft gelly.

Binders for tablets that can be included are acacia, methylcellulose, sodium carboxymethylcellulose, povidone, hydroxypropylmethylcellulose, sucrose, starch and ethylcellulose. Lubricants that can be used include magnesium stearate or other metallic stearate, stearic acid, fluid silicone, talcum, wax, oils and colloidal silica. Flavouring agents as peppermint, cherry flavour or similar, can also be used. Moreover it can be desirable adding a colouring agent to make more attractive the formulation or to make the product identification easier.

The compounds hereby mentioned which can be considered active whenever they are administered parenterally may be formulated for administration through the intramuscular, intratecal or intravenous route.

A typical formulation through intramuscular route consists of a suspension or a solution of the active ingredient in oil, such as peanut oil or sesame oil.

On the other hand, a typical composition for intravenous or intratecal administration may consist of a sterile water and isotonic solution which may contain the active ingredient and dextrose, or a mixture of dextrose and sodium chloride. Optionally a co-solvent, as polyethylene glycol, chelating agent, such as ethylendiamine tetraacetic acid and one antioxidant, e.g. sodium metabisulfite can be included in the formulation.

Alternatively, the solution might be dried by freezing/sublimation and then reconstituted through any suitable solvent just before its administration.

The hereof compounds which result to be active in rectal route administration can be formulated as suppository. A typical suppository formulation may consist of the active ingredient together with a binding agent and/or lubricant such as gelatine or cocoa butter or other wax or vegetable fat or low melting synthetic agents.

The hereof compounds which are active in topic administration can be formulated in the transdermal formulation or transdermal delivery system (dermal patches). These compositions include, for example, coating, reservoir of the active ingredient, a control membrane, dressing and contact adhesive. These dermal patches are likely to be used to provide a continuous or discontinuous infusion of the compounds described in this invention within controlled quantities. Production and use of dermal patches for releasing therapeutics are well known in the art. See, e.g. U.S. Pat. No. 5,023,252, issued 11 Jun. 1991.

The active compound has its effects among a broad range of dosages and it is generally administrated in an efficient pharmaceutical amount. The dosage which shall be administrated is supposed to be decided by the physician in charge, especially in consideration of relevant circumstances, as pathology involved, the route of administration adopted, the actual compound which is to be administrated and its relative activity, age, weight and any kind of patient's reaction to the drug; lastly, it is worth considering the severity of patient' s symptoms. Proper dosages may vary between 0.01-100 mg/kg/day, best 0.1-50 mg/kg/day. For what concerns a 70 Kg average man the appropriate dosage is to be 0.7 mg to 7 g per day, or more preferably 7 mg to 3.5 g per day.

Any other formulation suitable for using the present invention may be found in Remington's Pharmaceutical Sciences, Mace Publishing Company, Philadelphia, Pa. 17^{th} edition (1985).

The examples hereby provided mean to help an easier comprehension of the object and the utility of this invention, and shall not be considered as limits to such invention in any way.

### EXAMPLES

### Ex. 1: 3,3-Bis-(4-hydroxyphenyl)3H-naphtho[1,2-c]furan-1-one (Compound 1) e 3,3-Bis-(4-hydroxyphenyl)-1H-naphtho[1,2-c]furan-3-one (Compound 6)

These compounds were obtained through a condensation reaction between the 1,2-naphtahlic anhydride and the corresponding phenols in presence of stannum chloride as reported in M. Hubacher, J. Am. Chem. Soc., (1944), 66, 255).

The purification of the products was obtained by using a flash chromatography on silica gel using an eluent mixture of dichloromethane/methanol 98/2 v/v.

Both compounds 1 and 6 were obtained with a yield of 15%.

Compound 1, NMR (ppm): 8.35 d, 8.05 d, 7.94 d, 7.84 t, 7.69 t, 7.31 d, 6.99 d.

Compound 6, NMR (ppm): 8.35 d, 8.30 d, 8.05 t, 7.95 d, 7.69 t, 7.41 t, 7.31 d, 6.99 d.

### Ex 2: 3,3-Bis-(3-bromo-4-3,3-Bis-(3-bromo-4-hydroxyphenyl)-3H-naphtho[1,2-c]furan-1-one (Compound 4)

A solution of compound 1 (0.05 g; 0.14 mmol) was dissolved in dichloromethane (3 ml), then bromine (0.28 mol), previously dissolved in dichloromethane, was added thereto dropwise under stirring at room temperature. The reaction has been carried on for 5 hours and finally the solvent was removed by evaporation.

The resulting crude was thus purified by flash chromatography on silica gel using an eluent mixture consisting of dichloromethane/methanol 98/2 v/v to give the compound 4 (yield: 24) NMR (ppm): 8.38 d, 8.32 d, 8.07 d, 7.96 d, 7.87 t, 7.74 t, 7.60 d, 7.32 d, 7.15 d, 7.00 d.

### Ex 3: 3,3-bis-(3,5-bromo-4-hydroxyphenyl)-3H-naphtho(1,2-c)furan-1-one (Compound 5)

To a solution of compound 1 (0.05 g; 0.14 mmol, Example 1) dissolved in ethanol (2 ml), a bromine excess (0.7 ml) was added dropwise, followed by stirring at room temperature for 16 hours. Afterwards, the solvent was removed by evaporation and the resulting crude was thus purified by silica gel flash chromatography using an eluent mixture consisting of cyclohexane/ethyl acetate 80/20 v/v to give the compound 5 (yield: 64).

NMR (ppm): 8.34 d, 8.28 d, 8.14 d, 8.12 d, 7.84 t, 7.79 s, 7.75 t.

### Ex 4: 3,3-bis-(3-iodium-4-hydroxyphenyl-3H-naphtho(1,2-c)furan_{—}1_one (compound 3)

To a suspension of compound 1 (0.05 g; 0.14 mmol, Example 1) in glacial acetic acid (2.5 ml), an iodinechloride solution (0.65 mmol) is added dropwise, which was previously dissolved in glacial acetic acid, followed by stirring at room temperature overnight. Lastly, the solvent was removed by evaporation and the resulting residue was thus purified by silica gel flash chromatography using an eluent mixture consisting of dichloromethane/methanol 98/2 v/v to give the compound 3 (yield: 71.2).

NMR (ppm): 8.40 d, 8.34 d, 8.08 d, 7.97 d, 7.88 t, 7.77 t, 7.54 d, 7.37 dd, 7.20 d.

### Ex. 5: 3,3-Bis-(3-chlorine-4-hydroxyphenyl)1H-naphtho(1,2-c)furan-3-one (compound 7) and 3,3-Bis-(3-fluorine-4-hydroxyphenyl)1H-naphtho(1,2-c)furan-3-one (compound 8)

A mixture consisting of 1,2-naphthalic anhydride (0.5 g; 2.5 mmol), the relevant phenol (5.0 mmol) and of a catalytic amount of sulphuric acid was stirred at the temperature of 180° C. degrees for five hours. Then, the hereof mixture was cooled at room temperature and after adding water (to the reaction solution) the mixture was extracted with dichloromethane (3×30 mL). The organic phase was thus dehydrated by sodium sulphate and the solvent was dried off.

The resulting residue containing the final product was purified by silica gel flash chromatography using an eluent mixture consisting of dichloromethane/methanol 95/5 v/v to give the compound 7 (yield: 8) and the compound 8 (rate: 7).

Compound 7, NMR (ppm): 9.14 d, 8.50 d, 8.26 d, 3.02 d, 7.95 t, 7.85 t, 7.54 d, 7.37 dd, 7.20 d.

Compound 8, NMR (ppm): 9.14 d, 8.52 d, 8.29 d, 8.02 d, 7.97 t, 7.87 t, 7.29 dd, 7.22 dd, 7.17 dd.

### Ex 6: 33,3-Bis-(4-hydroxyl-5-isopropyl-2-methylphenyl)-3H-naphtho(1,2-c)furan-1-one (compound 2)

A mixture consisting of 1,2-naphthalic anhydride (0.5 g; 2.5 mmol) and thymol (5 mmol) was dissolved in tetrachloroethanol (25 ml), and aluminium chloride was added thereto (0.67 g; 5 mmol) in small quantities at room temperature. The suspension was then heated at 180° C. under agitation for 72 hours. The hereof mixture, still hot, was then decomposed with ice and dichloromethane was added thereto. Afterwards the organic phase was separated, dehydrated with sodium sulphate and dried off. The resulting residue was then purified by silica gel flash chromatography using an eluent mixture made of cyclohexane/ethyl acetate 70/30 v/v to give the compound 2 (yield: 6).

NMR (ppm): 9.50 s, 8.35 d, 8.30 d, 7.80 t, 7.70 d, 7.58 t, 7.05 d, 6.90 d, 6.60 d, 6.53 d, 3.22 m, 1.72 s, 1.10 t.

### BIOLOGICAL EXAMPLES

As examples of biological activity related to the compounds belonging to this patent, are successively described the enzymatic inhibition constants for compounds 1-8 towards TS isoenzymes (LcTS, EcTS, CnTS, hTS), let alone human DHFR.

As far as it is concerned for compounds 5 and 8 here is reported what turned out from sensitivity proofs towards bacterial multiresistant stocks coming from clinical isolated cases belonging to patients in antibiotic therapy.

As regards compounds 5, the result of the citotoxicity assay is reported

### ENZYMOLOGICAL TESTS

The strains and the plasmids were provided by doc. D. V. Santi of San Francisco University, California.

Enzyme LcTS purification: the method followed to purify the thymidylate synthase of Lactobacillus Casei is a modification of well-known processes (Maley G F, Maley F; J. Biol. Chem., (1988) 263, 7620-7627). Materials and methods: phosphocellulose (WAHTMAN 11); BIOGEL HAP (BIORAD); buffer 0.1 M KxHyPO4 (pH 7.00); buffer 1 M KxHyPO4 (pH 6.80); buffer W1 0.01 M (pH 6.80 ) (KxHyPO4 1M, 0.01 M EDTA ); buffer W2 (W1 0.01 M, 0.1 M KCl); buffer W3 (W1 0.01 M, 0.3 M KCl). Process: The TS, coming form Lactobacillus Casei, was prepared from a synthetic plasmid (pSCTS9), afterwards inoculated in Escherichia Coli (χ2913 ) cells.

Two litres of broth, each one containing 100 μg/mol of ampicillin, were grafted with nearly 40 mol of a transformed E. coli culture and were left growing in thermostat at 37° C. for 16 hours. Through centrifugation the cells contained in the broth have been divided and the so obtained pellet has been stored at −80° C.

The breaking of the cells has been performed manually using pestle and mortar in the presence of allumine. The cells residues have been removed through centrifugation at 11000 rpm for 50 minutes.

The raw extract (25 mL) has been loaded in the phosphocellulose column using a peristaltic pump at a speed of 1 mL/min. The column has been washed previously with 100 mL of W3 buffer and then with a same amount of W2 buffer. When phosphocellulose column has been linked with hydroxylhapatite column, the two columns have been washed with 100 mL of W3 buffer, at a speed of 1 mL/min. The greater conductivity of W3 buffer should let the enzyme be detached from the first column and move to the second. The enzyme has been after eluted with 80 mL of a KxHyPO4 tampon with a linear gradient that goes to 80 at 400 μM.

Fractions that resulted active relating to biological activity have been put together (8 mL) and then concentrated and equilibrated with a pH=6.8 buffer, composed by KxHyPO4, 20 mM and EDTA (ethylenediaminetetraacetic acid) 0.5 mM, using AMICON ultrafiltration system (centriprep 30) that has a YM-40 membrane unit.

The purified enzyme has been stocked at −80° C. Kinetic analysis made in stationary conditions has shown a Km for the methylenetetrahydrofolate (MTHF) of 12.8 μM and a Kcat of 2.6 sec⁻¹. Data related to the biological activity have been executed preparing a reaction mixture composed by TES buffer, dUMF 20 μM, MTHF 140 mL of each fraction eluted from the column; in these conditions the initial speed of the enzymatic reaction has been measured.

The Km measure has been made through assays of enzymatic activity in stationary conditions. These assays consist in measuring the initial speeds of the enzymatic reaction in presence of variable and growing concentrations of the substrate (MTHF): the reaction mixture is composed by enzyme, dUMF 120 μM, MTHF at a variable concentration that ranges form 3 to 140 μM. The enzyme concentration (9.1 μM) has been obtained through spectrophotometric method considering ε=125600 at λ=278 nm. As showed by SDS PAGE analysis (gel electrophoresis on sodium dodecyl sulphate-polyacrylamide), the enzymatic purity resulted greater than 95%. The purified enzyme has been stored at −80° C. in phosphate buffer 10 mM, 0.1 mM EDTA, at a 7.0 pH.

Enzyme EcTS purification: the used process is a modification of well-known procedures (Ahrweirer P M, Frieden C; J. Of Bacteriology (1988), 3301-3304. At the end the process various fractions obtained were pooled in buffer 25 mM KxHYPO4, 20 mM β-mercaptoethanol are dialyzed and finally stored at −80° C.

Kinetic analysis made in stationary conditions has shown a Km for MTHF of 7.54 μM.

Instances relating to enzymatic activity consist in measuring the initial speeds of enzymatic reaction mixture is composed by TES buffer, enzyme, dUMF 120 μM, water and MHTF at a concentration that goes from 2 to 64 μM. As showed by SDS PAGE analysis (gel electrophoresis on sodium dodecil sulphate-polyacrylamide), enzymatic purity resulted higher than 95%.

Purified enzyme has been stored at −80° C. in phosphate buffer 10 mM, 0.1 mM EDTA at 7.0 pH.

Human TS Purification

The hereof purification used is a modification of known procedures (Gourley D G, Luba J., Hardy L W, Beverly S M, Hunter W N; Acta Crystallography (1999), D25, 1608-1610 ).The fractions that showed enzymatic activity were pooled and were dialyzed with 10 mM KxHyPO4 (pH 7.5), 0.1 mM EDTA to decrease the concentration of KxHyPO4 and than favour the storage and the concentration through centriprep with a 30 Amicon membrane. The kinetic analysis in steady state conditions showed a Km for the folate substrate of 4.787 μM, while the k_{cat} is 0.07 sec-1. The enzymatic assays for the measurements of the Km consist in measuring the initial rate of the enzymatic reaction in presence of variable and increasing substrate (methylenetetrahydrofolate) concentration. The reaction mixture is made of TES buffer, enzyme, dUMP 120 μM, water and MTHF in concentration from 4 to 128 μM. As showed by the SDS PAGE analysis the enzymatic purity resulted higher than the 95%. The purified enzyme has been stored at −80° C. in phosphate buffer 10 mM, 0.1 mM EDTA at pH7.0.

Human DHFR Purification

The hereof purification used is a modification of known procedures (Pedersen-Lane J, Maley G F, Chu E, Maley F; Protein expression purif. (1997), 10, 256-262). hDHFR has been prepared form a plasmid (p593/M15) and successively inoculated in Escherichia Coli (χ2913) cells. The fractions that showed enzymatic activity were pooled and were dialyzed with 10 mM KxHyPO4 (pH 7.5), 0.1 mM EDTA to decrease the concentration of KxHyPO4 and than favour the storage and the concentration through ultrafiltration AMICON (centriprep 30) with membrane unit YM-30.

The purified enzyme has been stored at −80° C. The kinetic analysis showed a Km for the dhydrofolic acid (FAH2) of 1.944 μM. The enzymatic assays for the measurements of the Km consist in measuring the initial rate of the enzymatic reaction in presence of variable and increasing substrate (FAH2). The reaction mixture is made of TES buffer, enzyme, NADPH 120 μM, water in concentration ranging from 5 to 160 μM and from 1.2 to 40 μM.

Enzymatic inhibition assay: The compounds hereby analyzed were previously dissolved in DMSO. The inhibitor concentrated solution was obtained by dissolving 2 mg of compound in 1 mL of DMSO. Next, solutions thus obtained were successively preserved and the culture was continued in a refrigerating machine at 20 C. degrees, and the stability of their working conditions was constantly controlled. The spectrophotometrical determinations of enzymatic reaction kinetics were carried out using a Parkin-Elmer λ16 spectrophotometer, provided with a multicell system which was maintained at the constant temperature of 20 C. degrees, with a HAAAKE F3C thermostatted bath.

The elaboration of the data was carried out using Kaleidagraph 3.0; this software was provided by Macintosh (Adelbeck, Software Reading, Pa., 1989, version 2.3). The measurement of Ki (inhibition constant) was obtained through enzymatic inhibition essays carried out under stationary conditions. The hereof assays concern the measurement of the enzymatic reaction initial speeds, in the presence of increasing concentrations of inhibitor The reaction mixture consisted of: 0.07 μM TS enzyme with specific activity 2,3-3,5U/mg, dUMP 110-120 μM, MTHF concentrations which may vary from 0.2 to 50-80 μM, depending on the compound solubility and activity. The enzyme is added finally. The assay aiming at controlling TS enzymatic activity is repeated at regular intervals. It is furthermore assumed that inhibitors behave as competitive inhibitors with respect to MTHF and have a similar behaviour. In this case inhibition constants were obtained through a non-linear regression analysis, which utilized of the least squared method, of the enzymatic reaction initial speeds relating to the inhibitor concentration; equation was the following:

Vi=Vmax*(1−([I])/([I]+(Ki*(1+([s]/Km))))), where

Vi=enzymatic reaction initial velocity, Vmax=enzymatic reaction maximum velocity, I=inhibitor concentration, Ki=inhibition constant, S=substrate concentration, Km=Michaelis-Menten.

Using this equation it is possible to obtain Ki making use of a minimal amount of experimental data. The specificity index relating to each of the compounds (Bio. Med. Chem. ) refers to the relation between Ki_{hTS}/Ki_{other enzyme}.

Examples of enzymatic inhibition for the compounds described in the present invention are reported in Table 1.

From the analysis of inhibition data given in Table 1, compounds 1, 3-5, 7 and 8 in the present invention result to be specific inhibitors for bacterial TS, whereas they don't seem to have any inhibition property for what concerns human TS.

Microbiological Assays

The anti-bacterial properties of the compounds hereby described were evaluated towards: a) bacterial native strains comprehending gram-positive and gram-negative bacteria.

(ATTC or native strain); b) 23 isolated bacterial strains that have been clinically proved to be multiresistant at least to 18 antibiotic compounds currently used, likewise Vancomycin, and have resulted to be constituted of Staphylococcus epidermidis (11 strains), Staphilococcus aureus (4 strains), Staphylococcus haemoliticus (2 strains), Enterococcus faecium (4 strains) and Enterococcus gallinarum (2 strains).

Sensitivity Assay.

The bacterial cultures were obtained by sowing bacteria in Mueller-Hinton (Difco) stirring at 37 C for 24 hours, and were then diluted, after reaching an appreciable level of exponential growth, using a growth plot in order to get a 1×10 CFU/mL inoculum.

Following, the obtained cultures were put in contact with growing concentrations of the present compound. Furthermore, it was measured the minimal concentration inhibiting the growth (MIC) of each of these compounds. In Table 2 several MIC obtained by different bacterial pathogenic strains have been reported that were treated using compounds of the present invention and were also compared with those obtained through ciprofluoxacin (cpx).

Citotoxicity Assay

Aiming at evaluating the citotoxicity level, the compounds included in the present invention were submitted to the MTT assay, by using VERO cells on MEM soil/ground which was previously added with FCS %), penicillin (1%) (50 U/ml), streptomycine (50 μg/ml) e L-glutamine (1%) according to the Ishioka method (1988). Afterwards the cultures have been observed for 2 days through microscope at indirect light, and the living cells have been quantified in a Burker camera using the Tripan blue exclusion assay.

Later several increasing concentrations of the analyzed compound were added thereto and, after a 24-hour incubation period, the mitochondrial dehydrogenase activity was measured in the living cells (MTT assay) (Mosmann, T. 1983), by using a sample of the hereof compound which had not been previously as a comparison. The MTT assay results for Compound 5 were reported in Table 3.

The values of the MIC (reported in Table 2) clearly demonstrate that Compound 5 results to be active on the major part of the multiresistant strains taken into consideration. Particularly, the lowest MIC (0.5-1)were registered on S. epidermidis strains. It is worth considering that the S. epidemidis strain resistant to 17 antibiotics (among them betalactams, macrolides, antibiotic aminoglicosides) have resulted to be sensitive to 0.5 mg/L Compound 5.

These data were obtained by taking into consideration the S. epidermidis growth percentage in presence of a different concentrations compound.

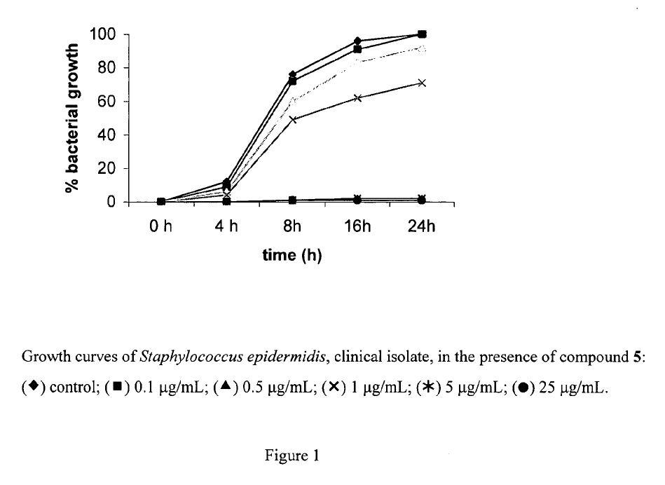

In FIG. 1 the growth curve is reported in percentage of S. epidermidis alone (control) and in presence of variable concentrations of Compound 5.

No inhibitory effect has been noticed at 0.1 mg/L, the antibacterial activity becomes evident after a 8 hours period with a 0.5 mg/L concentration. The best result was obtained using a 1 mg/L concentration of the present compound; this quantity was proved not to be citotoxic (Table 3). An antibacterial effect was noticed in higher concentrations

TABLE 1
Enzyme inhibition activity.

| | | | | | | | | | SSI hTS |
|---|---|---|---|---|---|---|---|---|---|
| | LcTS | EcTS | CnTS | hTS | hDHFR | SI LcTS | SI EcTS | SI CnTS | vs |
| | Kᵢ (μM) | Kᵢ μM) | Kᵢ (μM) | Kᵢ (μM) | Kᵢ (μM) | vs hTS | vs hTS | vs hTS | hDHFR |
| 1 | 11 | 4.4 | 0.4 | >>132 | 110 | 12 | 30 | 330 | 1 |
| 2 | 1.4 | 0.3 | 0.6 | 0.4 | 35 | 0.3 | 1 | 1 | 88 |
| 3 | 2.0 | 1.5 | 4.5 | >>245 | 3.9 | 123 | 163 | 54 | 0.01 |
| 4 | 6.6 | 0.9 | 2.7 | >>132 | 33 | 20 | 147 | 49 | 0.2 |
| 5 | 1.7 | 8.5 | 13 | >>245 | 69 | 144 | 29 | 19 | 0.3 |
| 6 | N.I. | N.I. | N.I. | N.I. | 13 | | | | |
| | (10 μM) | (10 μM) | (10 μM) | (10 μM) | | | | | |
| 7 | 3.9 | 1.2 | 8.1 | 34 | 35 | 9 | 28 | 4 | 1 |
| 8 | 1.4 | 1.4 | 3.5 | >>132 | 109 | 94 | 94 | 38 | 1 |
| CB3717 | 0.06 | 0.06 | | 0.03 | | 1 | 1 | | |

TABLE 2
Minimum inhibitory concentration (MIC)(μg/mL).

| | Compounds | | |
|---|---|---|---|
| Strains | 5 | 8 | cpx |
| Enterococcus faecalis | 2.5 | 25 | 12.5 |
| ATCCC 29212 | | | |
| Escherichia coli 256 | >25 | >50 | >25 |
| Escherichia coli 292 | >25 | >50 | >25 |
| Listeria monocytogenes 3 | 2.5 | 25 | 2.5 |
| Listeria monocytogenes 4 | 2.5 | 25 | 1 |
| Listeria monocytogenes 5 | 2.5 | 25 | 2.5 |
| Listeria monocytogenes 6 | 2.5 | 25 | 2.5 |
| Listeria monocytogenes 7 | 2.5 | 25 | 1 |
| Listeria monocytogenes 8 | 2.5 | 25 | 1 |
| Listeria monocytogenes | 2.5 | 25 | 2.5 |
| ATCC 4428 | | | |
| Staphylococcus aureus 341 | 2.5 | 25 | 1.2 |
| Staphylococcus aureus 343 | 2.5 | 25 | 0.5 |
| Staphylococcus aureus K28 | 2.5 | 25 | 0.5 |
| Staphylococcus aureus | 2.5 | 25 | 0.5 |
| ATCC29213 | | | |
| Staphylococcus | 2.5 | 25 | 2.5 |
| haemolyticus ATCC 2997 | | | |
| Staphylococcus | 1 | 25 | 1 |
| saprophiticus ATCC15305 | | | |
| Citrobacter 224 | >25 | >50 | >25 |
| Streptococcus 42 | 2.5 | 25 | >5 |

TABLE 3
Results of MTT test on VERO cells. Data are expressed as
percentage of cellular growth. Control vessels are without
compounds.

| | | Compounds |
|---|---|---|
| Concentration (mg/L) | Control | 5 |
| 50 | 100 | >100 |
| 25 | 100 | 100 |
| 12.05 | 100 | 100 |
| 6.25 | 100 | 100 |
| 3.12 | 100 | 100 |
| 1.56 | 100 | 100 |

1. A compound of the general formula (I) 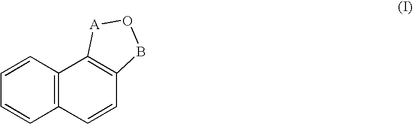
Wherein: A and B stand for C═O, a formula G molecular fragment 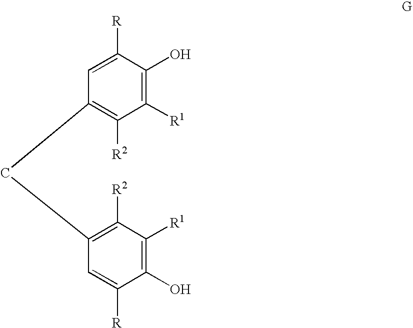
Wherein: R, R¹ and R² each represent:
(a) hydrogen;
(b) C₁₋₁₂ alkyl linear or branched chain, which can contain from 1 to 3 double or triple bonds and, if needed, can be substituted by/with one, two or three substituents such as fluorine, chlorine, bromine, iodine, NO, NR; wherein R may represent hydrogen, C₁₋₁₂ alkyl.
(c) halogen, including fluorine, chlorine, bromine, iodine,
(d) NO, NR, SH, SO HR groups, wherein R is the same as above defined, with the proviso that when A is C═O, so B is G, and when A is G, as a consequence B is C═O; in consideration of the fact that the hereof general formula compound is specified, if needed, through a regioisomeric (Iα)
or (Iβ) form:

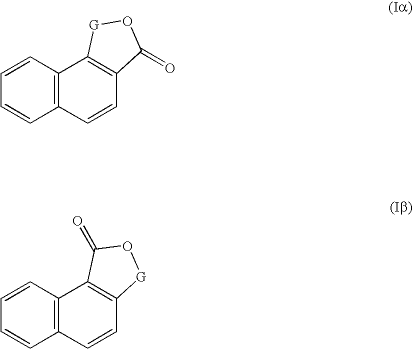

2. A method of preparation of a compound of the general formula (I) according to claim 1; said preparation method providing a reaction between 1,2-napthalic anhydride and a phenol compound, having the following formula; 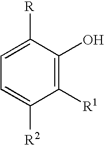
separating the resulting mixture of regioisomeric compounds of Formula I into the single regioisomeric compounds of formula (Iα) and (Iβ); further converting, if desired, a regioisomeric compound of Formula (Iα) in another compound of formula (Iα) or a regioisomeric compound of formula (Iβ) in another compound of formula (Iβ).

3. A compound of formula (I) according to claim 1, where said compound is:
3,3-Bis-(4-hydroxyphenyl)-3H-naphtho[1,2-c]furan-1-one (Compound 1)
3,3-Bis-(4-hydroxy-5-isopropyl-2-metilphenyl)-3H-naphtho[1,2-c]furan-1-one (Compound 2).
3,3-Bis-(3-iodo-4-hydroxyphenyl)-3H-naphtho[1,2-c]furan-1-one (Compound 3).
3,3-Bis-(3-bromo-4-hydroxyphenyl)-3H-naphtho[1,2-c]furan-1-one (Compound 4).
3,3-Bis-(3,5-bromo-4-hydroxyphenyl)-3H-naphtho[1,2-c]furan-1-one (Compound 5).
3,3-Bis-(4-hydroxyphenyl)-1H-naphtho[1,2-c]furan-3-one (Compound 6)
3,3-Bis-(3-chloro-4-hydroxyphenyl)-1H-naphtho[1,2-c]furan-3-one (Compound 7)
3,3-Bis-(3-fluoro-4-hydroxyphenyl)-1H-naphtho[1,2-c]furan-3-one (Compound 8).

4. Use of a compound in accordance with claim 1 in order to inhibit particularly the bacterial thymidylate synthase.

5. Use of an efficient quantity of a compound in accordance with claim 1 in order to prepare a medicament for the treatment, the prevention or the prophylaxis of an infectious pathology caused and/or supported by bacteria; said treatment being executed or simplified by inhibiting thymidylate synthase activity of pathogenous bacteria, including, (but not restricting to), Enterococcuc faecalis, Staphilococcus aureus, Criptococcus neoformans, Pneumocistis carini Listeria monocytogenes, fungi, parasites in a mammal, particularly human beings.

6. A pharmaceutical composition including a quantity of a compound that will be effective for the treatment, the prevention or the prophylaxis of an infectious pathology in accordance with claim 5, and a pharmaceutically acceptable carrier.
